(19) United States
(12) Patent Application Publication
(10) Pub. No.: US 2003/0050587 A1
(43) Pub. Date: Mar. 13, 2003

(54) Thigh and leg adjustable casting support

(76) Inventor: Darel E. Courser, Bakersfield, CA (US)

Correspondence Address: James M. Duncan, Klein DeNatale Goldner Cooper Rosenlieb & Kimball, P O Box 11172, Bakersfield, CA, 93389-1172, US

(21) Appl. No.: 09/948,366

(22) Filed: Sep. 7, 2001

(51) Int. Cl. A61F 5/00

(52) U.S. Cl. 602/32

(57) ABSTRACT

An adjustable support for supporting the upper part of the leg, the thigh, or the buttocks during an application of medical treatment to the leg, such as the application of a cast or an amputation is disclosed. The device provides a support of variable height which may be constructed according to dimensions and materials which may be sterilized inside available surgical sterilization equipment. The casting support comprises a base, a leg support frame, and locking means for selectively locking the support frame in any one of a plurality of positions relative to the base. The leg support frame is pivotally attached to the base. Leg support means, such as a roller, are attached to the leg support frame. The locking means selectively lock the support frame in any one of a plurality of positions relative to the base, as the leg support frame pivots about the base.

### BACKGROUND OF THE INVENTION

The present invention relates to an apparatus for positioning and supporting the leg of a patient during the application of a cast, and more particularly to an apparatus which quickly adjusts to the height required for the particular patient, including children. The present invention further particularly relates to an apparatus, which because of its portability, size and materials of construction, may readily be placed within a sterilizer, while still being strong enough to support the weight of an adult patient.

Under present surgical procedures, it is common to repair bone fractures within the leg by placing the patient under general anaesthesia and exposing the fracture for surgical repair, such as application of a surgical splint. Because of the incisions required by this practice, a sterile field is required. Following the surgical repair, a cast is applied to the leg while the patient remains under anaesthesia. Because the patient is under anaesthesia, it is important to complete the operation and casting procedure as quickly as possible. Depending upon the length of cast required, a support is usually required under the patient's thigh or buttocks so the cast may be applied with minimal movement applied to the leg.

A variety of devices are known for supporting a patient's limbs during the application of a cast. U.S. Pat. No. 3,143,110 describes a foot holder device for a cast table. The device includes both a structure to support the heel area of a foot and a sole plate which is slidably attached to the heel support structure. Among the objects of the device described in U.S. Pat. No. 3,143,110 are to provide a foot holding device which is light in weight, easy to clean after use, which can be mounted upon existing cast tables and which is easily and quickly adjustable into a variety of different positions to assist the doctor in placing and holding the foot in exactly the right position before and while the cast is being applied. Several other devices have been disclosed which provide support for the foot or lower part of the leg during application of a cast, including U.S. Pat. Nos. 3,908,643; 3,995,846; 4,261,348; 4,726,363; 4,941,463; and 5,242,140. All of these devices are generally designed to provide support to the lower portion of the leg but not to provide support under the thigh or buttocks.

The known devices for supporting a patient's upper thigh or buttocks during casting procedures are a cushion roll or a non-adjustable stand. The non-adjustable stand typically has metal sides which support a wooden roller, the roller usually covered with cast padding. In the case of young children, these devices are too high and often the surgical team must improvise a stand, using, for example, an IV fluid bag. However, valuable time may be lost in trying to adjust the leg to the correct elevation. Any unnecessary loss of time is undesirable because of the need to minimize the amount of time the patient is under anaesthesia and the need to make the operating room available for other patients. Often, the devices used for upper leg support are soiled with blood, iodine, plaster, urine or fecal material, so that the device must either be sterilized, disposed or isolated from the sterile environment Although the cast padding may be replaced, the metal supports and wooden roller of the known device are not sterile and must be isolated from the sterile field.

A device is required which provides stable support for a leg, where the elevation of the device is easily adjusted, and where the device is easy to clean and sterilize.

### SUMMARY OF THE INVENTION

The present invention is directed to a thigh and leg adjustable casting support which meets the needs identified above. The disclosed apparatus provides an adjustable support for supporting the upper part of the leg, the thigh, or the buttocks during an application of medical treatment to the leg, such as the application of a cast or an amputation. The device may also be used for the adjustable elevation of a patient's foot for application of a cylinder cast. The device may be constructed according to dimensions and materials which may be sterilized inside available surgical sterilization equipment.

One embodiment of the casting support comprises a base, a leg support frame, and locking means for selectively locking the support frame in any one of a plurality of positions relative to the base. The base has a front and a back. The leg support frame has a pivot end and a free end, where the pivot end is pivotally attached to the back of the base. Leg support means, such as a roller, are attached to the free end of the leg support frame. The locking means selectively lock the support frame in any one of a plurality of positions relative to the base, as the leg support frame pivots about the back of the base.

Another embodiment of the disclosed apparatus comprises a base, a leg support frame, leg support means, and locking means. The base has a front and a back, and a left support member and a right support member which are disposed in spaced apart facing relation. The left support member is attached to the right support member with a base plate. The left support member and right support member have a plurality of holes, each hole in the left support member having a corresponding hole in the right support member in spaced apart facing relation. The leg support frame comprises a pivot end and a free end, a left rail, and a right rail. The left rail and right rail are disposed in spaced apart facing relation. The pivot end of the leg support frame is pivotally attached to the back of the base. The left rail and right rail have a plurality of holes, each hole in the left rail having a corresponding hole in the right rail. A roller is disposed at the free end of the leg support frame, the roller fastened between the left rail and right rail. The apparatus has locking means for selectively locking the support frame in any one of a plurality of positions relative to the base, as the leg support frame pivots about the back of the base. The locking means comprise a first pin for inserting into a hole in the right support member and an adjacent hole in the right rail and a second pin for inserting into a hole in the left support member and an adjacent hole in the left rail.

These and other features, aspects, and advantages of the present invention will become better understood with regard to the following description, appended claims, and accompanying drawings.

### BRIEF DESCRIPTION OF THE DRAWINGS

FIG. 1 is an isometric view of the disclosed thigh and leg adjustable casting support.

FIG. 2 is a top view of the disclosed invention.

FIG. 3 is a back view of the disclosed invention.

FIG. 4 shows a side elevational view of the disclosed invention in a raised position.

FIG. 5 shows a side view elevational view of the disclosed invention in a lowered position.

FIG. 6 shows a side view of the disclosed invention showing one method of use.

### DETAILED DESCRIPTION OF THE EMBODIMENTS

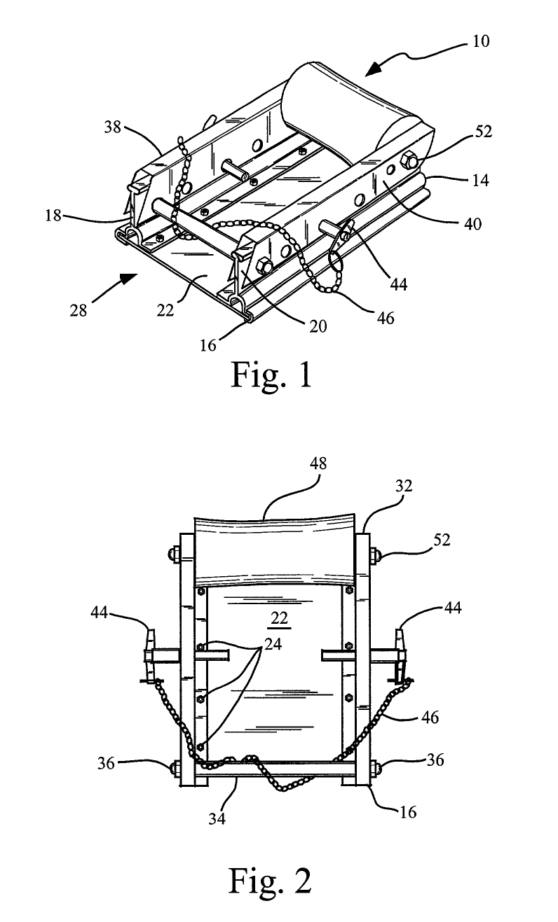

Referring now specifically to the drawings, FIG. 1 shows one embodiment of the thigh and leg adjustable casting support 10. The invention includes a base 12 which has a front 14 and a back 16. The base 12 may include a left support member 18, a right support member 20, and a base plate 22. The embodiment depicted in FIG. 1 shows the left support member 18 and the right support member 20 disposed in spaced apart facing relation, with the left support 18 member and right support member 20 generally parallel to one another. The left support member 18 and right support member 20 may be manufactured from carbon steel, aluminum, or other material capable of withstanding the sterilizer temperature, which may exceed 412 degrees Fahrenheit. The left support member 18 may be attached to the right support member 20 with base plate 22, which is fabricated from steel or alloy plate material. The left support member 18 and right support member 20 may be fastened to base plate 22 with various fastening means, such as stainless steel screws and cap nuts 24. According to the means employed for locking the device in the raised position, a plurality of holes 26 may be drilled in both the left support member 18 and the right support member 20, the holes 26 in the left support member 18 in approximate facing relation to the holes 26 in the right support member 20.

The thigh and leg adjustable casting support 10 has a leg support frame 28. The leg support frame 28 has a pivot end 30 and a free end 32, the pivot end 30 being pivotally attached to the back 16 of the base 12. One means of pivotally attaching the leg support frame 28 to the base 12 is to drill matching holes through the leg support frame 28 and the left support member 18 and right support member 20, so that a fastener may be inserted through the holes. As shown in FIG. 2, the fastener may be inserted through tube 34. If a threaded fastener is used, nuts 36 may be placed on either side of the fastener.

The leg support frame 28 may further comprise a left rail 38 and a right rail 40 disposed in spaced apart facing relation. The left rail 38 and right rail 40 may be fabricated from channel-type material so that the left rail 38 slides over left support member 18 and the right rail 40 slides over right support member 20 as depicted in FIG. 1. According to the means employed for locking the device in the raised position, a plurality of holes 42 may be drilled in both the left rail 38 and the right rail 40, the holes in facing relation. Depending on the desired elevation of the leg support frame 28, holes 26 in the left support member 18 and the right support member 20 should match with corresponding holes 42 in the left rail 38 and the right rail 40. Pins 44 may be used for locking the thigh and leg adjustable casting support 10 so that the leg support frame 28 is at the desire height. The diameter of holes 26 in the left support member 18 and the right support member 20 should match the diameter of the holes 42 in the left rail 38 and the right rail 40. It has been found that ⅜ inch diameter holes are acceptable, in which case pins 44 may be 5/16 inch pins with a spring-loaded ball lock at the end, which positively lock the device at the desired height. For convenience, pins 44 may be attached to the thigh and leg adjustable casting support 10 with security chains 46, which may be attached as desired to the device.

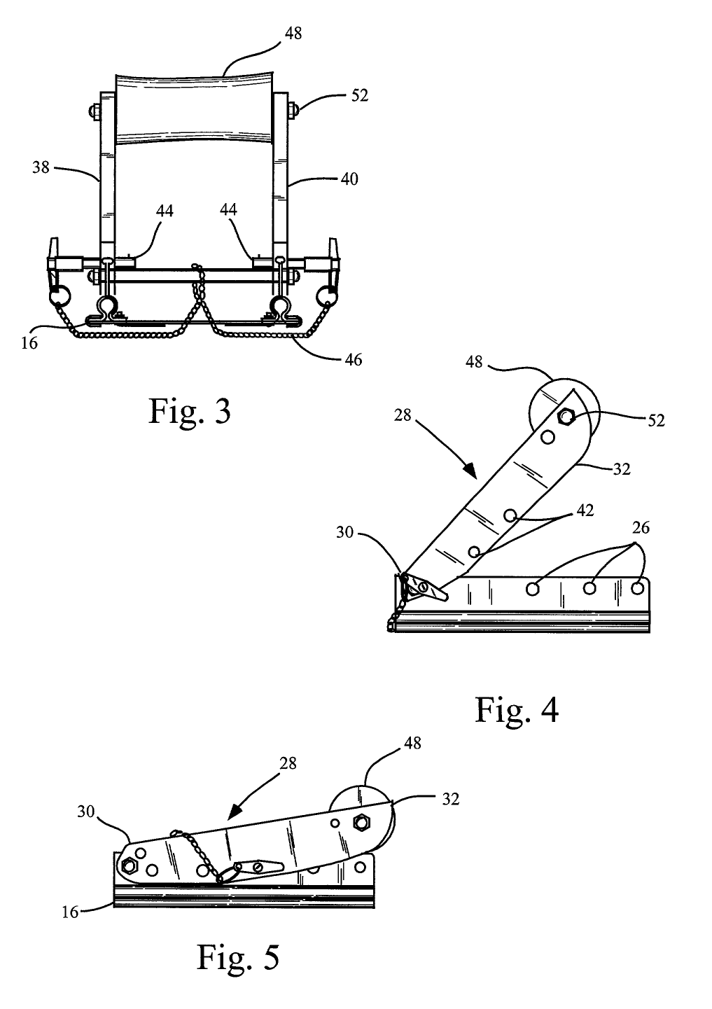

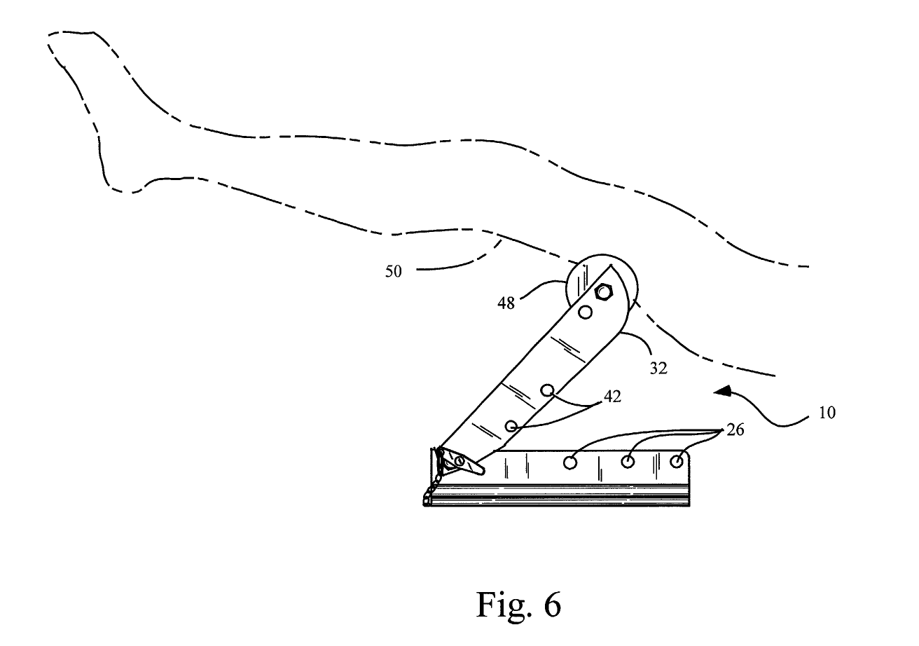

Leg support means, such as a roller 48, are attached at the free end 32 of the leg support frame 28. As shown in FIG. 6, the leg support means is the point of contact between the patient's leg 50 and the thigh and leg adjustable casting support 10. While the leg support means depicted in FIGS. 1 through 6 is a roller 48, those skilled in the art will appreciate that many different shapes of leg support means may be used to provide the necessary support to the patient's leg 50. However, because a roller 48 is free to roll within the leg support frame 28, movement of the patient's leg 50 over the thigh and leg adjustable casting support 10 is facilitated. The roller 48 is attached within the leg support frame 28 by fastening means such as a bolt with a rounded head and matching cap nut 52. A variety of different materials may be used to construct roller 48. A simple and inexpensive material is PVC pipe having approximate diameter of 3 inches, capped on either side with 2½ inch PVC plugs. However, if the thigh and leg adjustable casting support 10 is to be placed within a sterilizer, the roller should be constructed of a material capable of withstanding the temperature of the sterilizer, such as stainless steel, aluminum, or other metal or alloy.

Other means may be employed by those skilled in the art for selectively locking the leg support frame 28 in any one of a plurality of positions relative to the base 12, as the leg support frame 28 pivots about the back 16 of the base 12. For example, a ratcheting mechanism may be attached to the base 12 and the leg support frame 28 so that the device will be locked at different heights depending upon the position of the ratchet. In addition some known locking means will also act to raise or lower the leg support frame 28, and lock the leg support frame 28 at a particular position simply by ceasing the particular action required for raising or lowering. For example, a screw jack assembly or worm drive might be attached between the base 12 and the leg support frame 28, where the leg support frame 28 would be raised as the screw is tightened or lowered as the screw is loosened. The leg support frame 28 will remain locked at a particular position when rotation of the screw is stopped. A second example would be to employ a hydraulic cylinder and piston assembly, where the piston would extend as hydraulic fluid is pumped into the cylinder or contract as hydraulic fluid is released from the cylinder. The leg support frame 28 would be raised by the extending hydraulic piston and lowered when the piston contracts within the cylinder. The screw jack assembly and the hydraulic cylinder and piston assembly would allow almost infinite height positions over the range between the highest and lowest positions of the leg support assembly 28.

While the dimensions of the thigh and leg adjustable casting support 10 are not critical to its operation, it has been found that a convenient size for the device is an overall length of approximately 10 inches and a width of 8½ inches. The height of the leg support frame 28 in its lower-most position may be approximately 5 inches. The height of the leg support frame in its upper-most position may be approximately 10 inches. It has been found that if the device is constructed in approximately these dimensions, the device is easy to manipulate and fits within most sterilization units, but still provides a firm support for a patient's leg. For ease of cleaning, the entire device 10 may be chrome plated.

While the above is a description of various embodiments of the present invention, further modifications may be employed without departing from the spirit and scope of the present invention. For example, the size, shape, and/or material of the various components may be changed as desired. Thus the scope of the invention should not be limited by the specific structures disclosed. Instead the true scope of the invention should be determined by the following claims.

What is claimed is:

1. A thigh and leg adjustable casting support for supporting a leg for medical treatment comprising:
(a) a base;
(b) a leg support frame pivotally attached to the base;
(c) leg support means attached to the leg support frame; and
(d) locking means for selectively locking the leg support frame in any one of a plurality of positions relative to the base, as the leg support frame pivots about the base.

2. The casting support of claim 1 wherein the base further comprises a left support member and a right support member, disposed in spaced apart facing relation, the left support member attached to the right support member with a base plate.

3. The casting support of claim 1 wherein the leg support frame further comprises a left rail and a right rail disposed in spaced apart facing relation.

4. The casting support of claim 3 wherein the leg support means comprises a roller attached to the leg support frame, the roller disposed between the left rail and right rail and attached with fastening means.

5. A thigh and leg adjustable casting support for supporting a leg for medical treatment comprising:
(a) a base having a front and a back;
(b) a leg support frame having a pivot end and a free end, the pivot end pivotally attached to the back of the base;
(c) leg support means attached to the free end of the leg support frame; and
(d) locking means for selectively locking the leg support frame in any one of a plurality of positions relative to the base, as the leg support frame pivots about the back of the base.

6. The casting support of claim 5 wherein the base further comprises a left support member and a right support member, disposed in spaced apart facing relation, the left support member attached to the right support member with a base plate.

7. The casting support of claim 6 wherein the leg support frame further comprises a left rail and a right rail disposed in spaced apart facing relation.

8. The casting support of claim 7 wherein the leg support means comprises a roller attached at the free end of the leg support frame, the roller disposed between the left rail and right rail and attached with fastening means.

9. The casting support of claim 7 wherein the left support member and right support member have a plurality of holes, each hole in the left support member having a corresponding hole in the right support member in spaced apart facing relation, and the left rail and right rail having a plurality of holes, each hole in the left rail having a corresponding hole in the right rail.

10. The casting support of claim 9 wherein the locking means comprises a first pin for inserting into a hole in the right support member and an adjacent hole in the right rail and a second pin for inserting into a hole in the left support member and an adjacent hole in the left rail.

11. The casting support of claim 10 wherein the first pin and the second pin are attached to the casting support with security chains.

12. A casting support for supporting a leg for medical treatment comprising:
(a) a base comprising a front and a back, a left support member and a right support member, disposed in spaced apart facing relation, the left support member attached to the right support member with a base plate and the left support member and right support member having a plurality of holes, each hole in the left support member having a corresponding hole in the right support member in spaced apart facing relation;
(b) a leg support frame comprising a pivot end and a free end, a left rail and a right rail, the left rail and right rail disposed in spaced apart facing relation, and the pivot end pivotally attached to the back of the base, the left rail and right rail having a plurality of holes, each hole in the left rail having a corresponding hole in the right rail;
(c) leg support means comprising a roller disposed at the free end of the leg support frame, disposed between the left rail and right rail and attached with fastening means; and
(d) locking means for selectively locking the support frame in any one of a plurality of positions relative to the base, as the leg support frame pivots about the back of the base, the locking means comprising a first pin for inserting into a hole in the right support member and an adjacent hole in the right rail and a second pin for inserting into a hole in the left support member and an adjacent hole in the left rail.

13. The casting support of claim 12 which is manufactured from materials capable of withstanding a temperature in excess of 300 degrees Fahrenheit.

14. The casting support of claim 12 wherein the device is chrome plated.
